(19) United States
(12) Patent Application Publication
(10) Pub. No.: US 2009/0198339 A1
(43) Pub. Date: Aug. 6, 2009

(54) SYSTEMS AND METHODS FOR SPINAL FUSION

(75) Inventors: Jeffrey KLEINER, Denver, CO (US); Michael SERRA, San Diego, CA (US)

Correspondence Address: NuVasive;c/o CPA Global, P.O. Box 52050, Minneapolis, MN, 55402, US

(73) Assignee: NuVasive, Inc., San Diego, CA (US)

(21) Appl. No.: 12/367,464

(22) Filed: Feb. 6, 2009

Related U.S. Application Data
(60) Provisional application No. 61/026,608, filed on Feb. 6, 2008.
(60) Provisional application No. 61/028,886, filed on Feb. 14, 2008.
(60) Provisional application No. 61/105,384, filed on Oct. 14, 2008.

(51) Int. Cl. A61F 2/44; A61F 2/28; A61B 19/00

(52) U.S. Cl. 623/17.16; 623/17.11; 623/23.49; 128/898

(57) ABSTRACT

An implantable intervertebral fusion cage including a removable means for retaining material inside of the cage during implantation. Embodiments of the present invention are directed toward an implantable intervertebral fusion cage that includes at least one removable shield or veneer that is capable of retaining a surgically useful material, such as a spinal fusion-inducing material, inside of the fusion cage during implantation and/or until the shield or veneer is removed.

### CROSS REFERENCE TO RELATED APPLICATIONS

This is an nonprovisional application claiming the benefit of priority under 35 USC §119(e) from U.S. Provisional Patent Application Ser. No. 61/026,608, filed on Feb. 6, 2008, the complete disclosure of which is hereby incorporated by reference into this application as if set forth fully herein.

Additionally, the following commonly-owned and co-pending applications are incorporated by reference into this disclosure in their entireties: U.S. application Ser. No. 11/093,409, filed Mar. 29, 2005 and entitled “Systems and Methods for Spinal Fusion,” U.S. Provisional Application Ser. No. 61/028,886, filed Feb. 14, 2008 and entitled “Spinal Distraction and Implantation Assembly and Related Methods,” and U.S. Provisional Application Ser. No. 61/105,384, filed Oct. 14, 2008 and entitled “Spinal Distraction and Implantation Assembly and Related Methods.”

### FIELD OF THE INVENTION

The present invention relates to the field of medical devices and is directed toward an implantable intervertebral fusion cage that includes a removable means for retaining material inside of the cage during implantation.

### BACKGROUND OF THE INVENTION

Spondylosyndesis, or spinal fusion, is a surgical technique used to combine two or more vertebrae into a single, rigid working unit. This is typically achieved by introducing a supplementary bone tissue, such as an autograft or allograft, into the intervertebral space between two target vertebrae at the location that is typically occupied by an intervertebral disc. The supplementary bone tissue is then used in conjunction with the patient's natural osteoblastic processes in order to grow bone or osseous tissue between the two or more target vertebrae, which acts to fuse them together into the desired rigid unit. This procedure is used primarily to eliminate pain that is caused by abnormal motion of one or both of the target vertebrae; pain relief occurs by immobilizing the vertebrae themselves and preventing the abnormal motion. Alternatively, surgically implantable synthetic intervertebral fusion cages may be used to perform spinal fusion procedures.

When in use, a bone graft implant or synthetic fusion cage may be filled with an orthopedic matrix containing additional fusion-promoting material (FPM), for example including but not limited to calcium hydroxyapatite, bone morphogenic protein (BMP), demineralized bone matrix, collagen bone graft matrix (e.g. Formagraft®) and stem cell material (e.g. Osteocel®) or other fusion-promoting substance placed within the spaces of the implant. The implant is then implanted into a patient at the desired location along that patient's spine where it will serve to promote bone growth and, ultimately, fusion of the two target vertebrae. The fenestrations present in a typical intervertebral fusion cage allow the supplementary bone tissue to partially escape from the hollow interior of the cage and make sufficient contact with the target vertebrae, thereby promoting fusion of the target vertebrae through the fusion cage itself. Unfortunately, the fenestrations, which are essential for the proper functioning of the intervertebral fusion cages, also present a major problem during surgical implantation of the cage in that they are poor retainers of the FPM inside of the fusion cage during implantation. FPMs may vary in viscosity, however one example of a FPM typically used in conjunction with an intervertebral fusion cage is a viscous liquid that does not move as freely as water, however it is sufficiently liquid in form so as to readily move from, or leak out of, the interior chamber of the fusion cage during implantation. Since this material is intended to promote the formation of bony tissue inside of a patient's body, a leak of this material out of the fusion cage during implantation can lead to several undesirable complications, including the creation of bony structures or osseous tissue at a location away from the surgical site (e.g. heterotopic bone formation), nerve compression, and failure to properly fuse the area between the two target vertebrae.

It would be advantageous to provide a surgically implantable intervertebral fusion cage that is adapted to and/or capable of retaining the FPM inside of the cage during implantation and/or thereafter, until such time as it is desirous for the FPM to come into contact with the target vertebrae, thereby preventing the material from flowing from, or moving out of, the interior of the fusion cage during implantation and thereby significantly reducing, if not eliminating, the risks noted above.

### BRIEF SUMMARY OF THE INVENTION

The present disclosure provides for this type of fusion cage and therefore addresses and rectifies the problems associated with the prior art. Embodiments of the present disclosure generally relate to apparatus and methods of use of a FPM containment device via generally planar elements disposed on either side of a fusion implant having one or more apertures to receive fusion-promoting material.

Embodiments of the present disclosure provide for an implantable intervertebral fusion cage that is capable of retaining material, and particularly FPM, within the interior chamber of the fusion cage during use (e.g. during surgical implantation) via the use of retaining means that serve to hold the FPM inside of the fusion cage. Other embodiments of the present invention provide a means of freely and readily removing the retaining means from the fusion cage after surgical implantation, and/or at any other desired time. In some embodiments, the present invention also includes methods of using and/or surgically implanting fusion cages of the present invention. Although well suited for use in human patients, and although much of the discussion of the present invention is directed toward use in humans, advantages offered by the present invention may be realized in the veterinary and scientific fields for the benefit and study of all types of animals and biological systems. Additionally, although the fusion cages of the present invention are particularly well-suited for implantation into the spinal column between two target vertebrae, and although much of the discussion of the present invention is directed toward their use in spinal applications, advantages offered by embodiments of the present invention may also be realized by implantation at other locations within a patient where the fusion of two or more bony structures may be desired. As one of skill in the art will appreciate, the present invention has applications in the general field of skeletal repair and treatment, with particular application to the treatment of spinal injuries and diseases. It should be appreciated, however that the principles of the present invention can also find application in other areas, specifically where there is a desire to constrain added fluid material to particular regions. For example, the present invention finds application in methods where the objective is to confine added material to predetermined areas of interest and to prohibit the undesired translocation of such material until an operation is complete and/or until a predetermined later time.

As used herein, the term “veneer” has a particular meaning within the art, but should also be understood to generally relate to any structure that acts as a barrier to the undesired translocation of material, and in particular, additional fusion-promoting material (FPM), for example including but not limited to calcium hydroxyapatite, bone morphogenic protein (BMP), demineralized bone matrix, collagen bone graft matrix (e.g. Formagraft®) and stem cell material (e.g. Osteocel®) or other fusion-promoting substance placed within the spaces of the implant, whether it be synthetic or otherwise. Veneers can therefore be constructed of any suitable material, including resorbable material or non-absorbable material, dissolvable material, or other similar material and may have various desired physical attributes specifically adapted for particular applications, for example, being flexible, subject to having its physical properties modified by application of light, chemicals, porous, colored, radio-opaque, etc. Moreover, the use of the term “cage” has a meaning understood in the art but may also be understood to generally relate to any geometrical structure, especially an enclosing structure, such as a box, rectangle, cylinder, or other physical configuration, that is adapted to constrain or confine material for desired periods of time within predetermined physical parameters.

In accordance with at least one embodiment of the present disclosure, an implantable intervertebral fusion cage is provided comprising a frame with at least one wall defining a hollow interior, the wall having at least one, and preferably a plurality, of openings or fenestrations through it allowing for fluid communication between the hollow interior and the exterior of the cage, the wall further including at least one ridge located on an exterior surface of the wall that is configured to secure or hold one or more shields or veneers in place along the exterior surface of the wall so as to cover the at least one, and preferably a plurality, of openings. The ridge is configured so as to allow the veneer to be freely removed from and replaced onto the exterior surface of the wall. When the veneer is in place along the exterior of the cage and secured or held in place by the ridge, thereby at least partially covering the at least one opening, the veneer substantially prevents or precludes fluid communication between the hollow interior and the exterior of the cage. When the veneer is removed from the ridge and is no longer secured or held in place along the exterior surface of the wall, fluid communication is restored between the hollow interior and the exterior of the cage.

The intervertebral fusion cage of the present disclosure is typically geometrical in shape and/or design. In varying embodiments, the cage may be configured and/or shaped as a box, square, rectangle, tube, disc, rod, cylinder, cone, and/or cage and/or may also take on any one or more other shapes or configurations that may be useful in the fusion of two adjacent bony structures within a patient. Additionally, the intervertebral fusion cage of the present disclosure may be adapted to promote the fusion of the bony structures when situated between two bony structures of interest, or adjacent to them.

In accordance with other aspects of the present invention, an implantable intervertebral fusion cage is provided comprising a rectangular frame having a top wall, a bottom wall and four lateral walls, each lateral wall being connected to the top wall at a top edge and to the bottom wall at a bottom edge in such a way so as to form a hollow rectangular structure when the walls are interconnected. The top wall, bottom wall and the four lateral walls define a hollow interior of the cage when fully interconnected, and the top wall and the bottom wall each have at least one opening or fenestration through them allowing for fluid communication between the hollow interior and an exterior of the cage. The top wall and the bottom wall each further include at least one ridge located on the exterior surface of the top wall and the exterior surface of the bottom wall respectively, the ridges being configured to hold a first veneer in place along the exterior surface of the top wall and a second veneer in place along the exterior surface of the bottom wall. The ridges are configured to allow the first veneer and the second veneer to be freely removed from, and replaced onto, the exterior surface of the top wall and the exterior surface of the bottom wall, respectively. When the first veneer is located along the exterior surface of the top wall and the second veneer is in place along the exterior surface of the bottom wall of the cage, and held in place by a restraining means or preclusive barrier, which in one embodiment is the ridges, the first veneer and the second veneer block the at least one opening or fenestration and prevent fluid communication between the hollow interior and the exterior of the cage. When either the first veneer or the second veneer is, or both the first and second veneers are, removed from the at least one ridge, fluid communication is at least partially restored between the hollow interior and the exterior of the cage. In some aspects of this embodiment of the present disclosure, the rectangular frame of the cage is square in shape.

In accordance with still other aspects of the present disclosure, an implantable intervertebral fusion cage is provided comprising a cylindrical, tubular or conical frame having a main wall, a first end wall and a second end wall, each end wall being interconnected to the main wall so as to form a closed cylinder, tube or cone when the walls are fully interconnected. The main wall, first end wall and second end wall, when fully interconnected, define a hollow interior of the cage. The main wall has at least one, and preferably a plurality, of openings allowing for fluid communication between the hollow interior and the exterior of the cage. The main wall further includes at least one ridge located on an exterior surface of the main wall that is configured to secure or hold a veneer in place along the exterior surface. The at least one ridge is configured to allow the veneer to be freely removed from, and replaced onto, the exterior surface of the main wall. When the veneer is located along the exterior surface of the main wall and secured or held in place by the at least one ridge, the veneer substantially covers or blocks the at least one opening and substantially prevents fluid communication between the hollow interior and the exterior of the cage. When the veneer is removed from the at least one ridge, fluid communication is at least partially restored between the hollow interior and the exterior of the cage.

In accordance with still other aspects of the present invention, a method of surgically implanting an intervertebral fusion cage into a desired location within a patient is provided. In at least one embodiment of these aspects of the present invention, the method comprises first obtaining a cage having: (i) at least one wall defining a hollow interior of the cage, said wall having at least one opening or fenestration in it that allows for fluid communication between the hollow interior and the exterior of the cage; (ii) at least one constraining structure, such as a ridge, on an exterior surface of the wall that is configured to reversibly hold at least one veneer in place along the exterior surface of the wall, wherein the at least one ridge is configured to allow the at least one veneer to be removed from, and replaced onto, the exterior surface of the wall; and (iii) at least one removable veneer. The next step to the method includes the surgeon or user preparing the cage for surgical implantation by filling the hollow interior with a material that is capable of fusing two vertebrae, or other bony structures, together and placing the at least one veneer along the exterior surface of the cage such that the at least one veneer is secured or held in place along the exterior surface by the at least one ridge and wherein the at least one veneer substantially blocks the at least one opening or fenestration, thereby substantially preventing fluid communication between the hollow interior and the exterior of the cage and thereby retaining at least most of the material inside of the hollow interior. The next step of the method is to locate an appropriate site inside of the patient for implantation of the cage and create a surgical opening in the patient that is sufficient to accommodate the cage. Once the opening is created, the next step in the method is to surgically implant the cage into the patient in such a way that the fusion of the two vertebrae or other bony structures will occur upon exposure to the material. Thereafter, the at least one veneer is removed from contact with the ridge and thus the exterior surface of the wall, restoring fluid communication between the hollow interior and the exterior of the cage and allowing the material to move from the hollow interior to the exterior of the cage. Once these tasks are completed, the method is concluded by closing the opening in the patient

In accordance with still other embodiments of the present disclosure, a device is described that is directed to a spinal surgery graft containment device designed to contain the location of graft material in situ, or a method employing the same, including but not limited to a device or method that employs generally planar elements disposed on either side of a fusion implant having one or more apertures therein that can receive graft material.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIGS. 1 and 2 show top and end views, respectively, of an example of a fusion cage with ridges according to one embodiment of the present invention;

FIGS. 3 and 4 show top and side views, respectively, of an example of a veneer according to one embodiment of the present invention;

FIG. 5 illustrates an example of a ridge forming part of an intervertebral fusion cage according to one embodiment of the present invention;

FIG. 6 illustrates a close up of a removable veneer in contact with an external surface of a cage and held in place by a ridge of FIG. 5;

FIGS. 7 and 8 illustrate alternative examples of a ridge forming part of an intervertebral fusion cage according to one embodiment of the present invention;

FIGS. 9 and 10 illustrate an example of an intervertebral cage with a removable veneer placed along an external surface of the cage according to one embodiment of the present invention;

FIGS. 11 and 12 illustrate perspective and end views, respectively, of an example of a tubular or cylindrical cage with two removable veneers in contact with an external surface of the cage and held in place by two ridges according to one embodiment of the present invention;

FIGS. 13-15 illustrate multiple views of an example of an intervertebral fusion cage having belt ridges according to one embodiment of the present invention;

FIG. 16 is an exploded perspective view of an example of an intervertebral fusion cage and veneer combination according to an alternative embodiment of the present invention;

FIG. 17 is a perspective view of the fusion cage and veneer combination of FIG. 16 coupled together;

FIGS. 18-22 are multiple views of the fusion cage of FIG. 16;

FIGS. 23-26 are multiple views of the veneer of FIG. 16;

FIGS. 27-29 are multiple views of the fusion cage and veneer of FIG. 16, illustrating assembly of the combination;

FIGS. 30-32 are multiple views of the fusion cage and veneer combination of FIG. 16 coupled together;

FIGS. 33 and 34 are multiple views of the fusion cage and veneer combination of FIG. 16 associated with one example of an inserter according to an embodiment of the present invention; and

FIGS. 35-37 are multiple views of an alternative inserter for use with the fusion cage and veneer combination of FIG. 16 according to an embodiment of the present invention; and

FIG. 38 is a side view of the fusion cage and veneer combination of FIG. 16 coupled to the inserter of FIG. 35.

### DETAILED DESCRIPTION OF THE PREFERRED EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The spinal fusion implant and related methods disclosed herein boasts a variety of inventive features and components that warrant patent protection, both individually and in combination.

The present disclosure provides for an implantable intervertebral fusion cage that is capable of retaining material, and particularly FPM, within an interior chamber of the fusion cage during use and/or during surgical implantation via the use of retaining means that serve to hold the FPM inside of a hollow chamber of the fusion cage. The present disclosure also provides for a means of removing the retaining means from the implantable fusion cage upon implantation of the cage, or at any other desired time. The present disclosure therefore improves upon the surgically implantable intervertebral fusion cages generally present in the prior art by including at least one shield or veneer that is held along at least one exterior surface of an intervertebral fusion cage and that serves to at least partially, preferably at least substantially, and even more preferably completely, block the movement of material from the hollow interior chamber of the cage to the exterior of the cage until such time as it is desirous for such movement to occur.

The at least one veneer is freely removable and may be put into place and removed from the cage numerous times with no loss of function. The at least one veneer may be held in place along the exterior of the intervertebral fusion cage by at least one ridge, located along an external surface of the cage, that overlaps with at least a portion of at least one edge of the veneer so as to secure the veneer in place on the external surface and prevent the veneer from moving or migrating during use and/or implantation of the cage. The contact of the at least one ridge with the at least one veneer is therefore sufficient to hold the veneer in place during use and/or implantation of the cage, but is also operable to allow the veneer to be freely removed from, and/or replaced onto, the cage. It is intended in some embodiments that the veneer of the present disclosure be freely removable from the ridge by the user or health care provider implanting the cage, but that the at least one veneer be held in place by the at least one ridge sufficiently tightly so as to prevent any unintended movement or shifting during surgical implantation of the cage and therefore prevent fluid communication between the hollow interior chamber and the exterior of the cage. One having skill in the art will appreciate that the veneer may be a film, a laminate, a wedge, a section, a segment, a shim, a plug, a coating, a surface, a plate and a cover that is operable to engage at least one exterior surface of an intervertebral fusion cage, and operable to at least partially, preferably at least substantially, and more preferably completely, block the movement of material from the hollow interior of the cage to the exterior of the cage.

In accordance with at least some aspects of at least one embodiment of the present disclosure, a surgically implantable intervertebral fusion cage is provided. The fusion cage includes a frame with at least one wall defining a hollow interior within the frame, the at least one wall having sufficient structural stability so as to withstand surgical implantation in a patient and to withstand the physical stresses that it will encounter after implantation as the patient moves. Although not required in the present disclosure, in some embodiments, the walls that comprise the cage will be generally planar elements. Generally planar is meant to mean relating to, or lying in a plane. A plane is a three-dimensional surface where, for any two points, a straight line joining the two points will lie wholly inside the surface. One having skill in the art will appreciate that the generally planar elements may be any structure or device that is capable of spanning two lines in space, and may be made of a material and configured to be structurally capable of resisting deformation or substantial plastic deformation under compression forces. While it is not required for purposes of the present disclosure, in some embodiments the cage will have a plurality of walls defining the hollow interior such that the cage may take the shape of a rectangle, square, box, tube, cylinder, cone, polygon, or other shape suitable for implantation into a patient. The at least one wall has at least one, and in some embodiments a plurality, of openings, apertures or fenestrations through it that provide open access to the hollow interior of the fusion cage and that allow for fluid communication between the hollow interior of the cage and the exterior of the cage. These openings, apertures and/or fenestrations serve the purpose of allowing a FPM, which is inserted into the hollow interior prior to surgical implantation, to leave the hollow interior of the cage, make contact with the desired vertebrae or other bony structures, and promote the fusion of the two vertebrae or other bony structures. Further, one having skill in the art will appreciate that as used herein, an opening, aperture and/or fenestration through the wall may be a perforation, a slot, a gap, a hole, a cavity, a notch or other breach in the wall that enables fluid communication between the hollow interior of the cage and the exterior of the cage. Further, the openings, apertures and/or fenestrations need not necessarily permit an unobstructed straight path from a point in the hollow interior of the cage to a point in the exterior of the cage (e.g., the wall may be comprised of a plurality of layers, wherein each layer has at least one opening aperture and/or fenestration, and the openings, apertures and/or fenestrations are offset and non-overlapping).

The at least one wall may include at least one ridge located on an exterior surface of the at least one wall such that the at least one ridge is located on an exterior surface of the cage relative to the hollow interior. The at least one ridge functions to hold at least one veneer to the exterior surface of the cage and is preferably configured so as to hold the veneer against the exterior surface of the cage sufficiently tightly so as to at least substantially block the fluid communication between the hollow interior of the cage and the exterior of the cage while the veneer is in place. The at least one ridge is also configured so as to allow the veneer to be removed at any time it is desirous for the user to do so, such as after surgical implantation. Preferably, when the veneer is held in place along the exterior surface by the at least one ridge, the veneer serves to at least partially, and preferably completely, cover or block the at least one opening or fenestration in the wall, thus preventing fluid communication between the hollow interior and the exterior of the cage and thereby preventing the movement of material out of the hollow interior. Therefore, when the veneer is removed from contact with the ridge and the exterior surface of the cage, fluid communication is restored between the hollow interior and the exterior of the cage and material will be free to move from the hollow interior to the exterior of the cage.

In another embodiment of the present disclosure, the at least one wall may be comprised of a plurality of generally parallel, planar walls, each wall in the plurality having at least one opening, aperture and/or fenestration. The plurality of generally parallel, planar wills may be a laminate member. The resulting structure would have at least one interior wall and at least one exterior wall creating a substantially planar space between the two walls. The substantially planar space may be configured to hold at least one veneer against one of the exterior surface of the inner wall and the interior surface of the outer wall. The substantially planar space and the veneer are preferably configured so as to hold the veneer against one of the inner and outer wall sufficiently tightly so as to at least substantially block the fluid communication between the hollow interior of the cage and the exterior of the cage while the veneer is in place. As in the foregoing paragraph, the substantially planar space and the veneer are configured so as to allow the veneer to be removed at any time it is desirous for the user to do so, such as after surgical implantation.

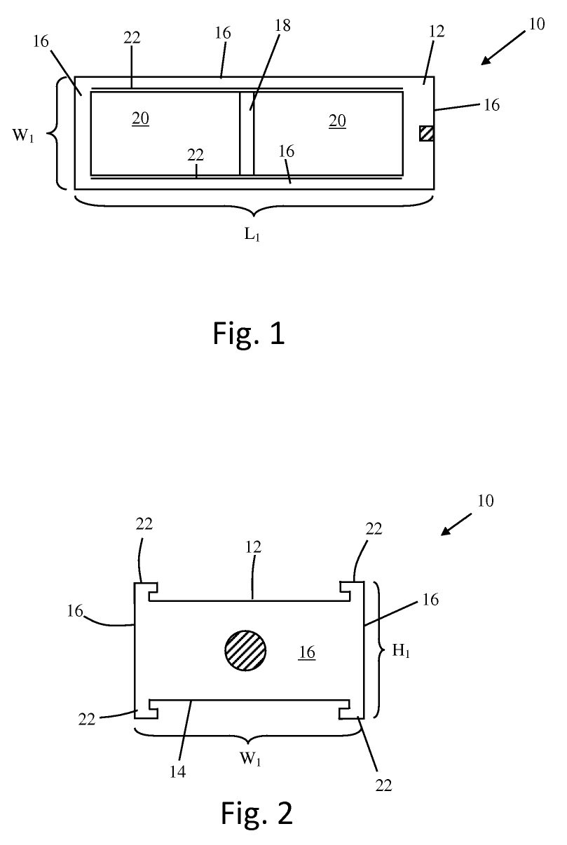

FIGS. 1 and 2 illustrate an example of an intervertebral fusion cage 10 according to one broad aspect of the present invention. The fusion cage 10 has a basic rectangular construction and, for the sake of simplicity, will be referred to herein as having a top wall 12, a bottom wall 14 and four lateral walls 16, which collectively form the shape of a rectangle in that each lateral wall 16 is connected to a single edge of the top wall 12 at a top edge of the lateral wall 16 and the bottom edge of the lateral wall 16 is connected to a single edge of the bottom wall 14 in such a way that the top wall 12, bottom wall 14 and four lateral walls 16, when fully interconnected, create a shape that is substantially rectangular or square-shaped and also define a hollow interior to the cage. In some embodiments, additional walls 18 may be present, for example within the interior chamber of the cage in order to create at least two separate hollow interior chambers within the cage and the ridges and veneers 24 of the present disclosure will operate identically with these embodiments with no loss of function whatsoever.

The intervertebral fusion cage 10 has a height H₁ ranging from about 8 mm to about 18 mm, a width W₁ ranging from about 10 mm to about 12 mm, and a length L₁ ranging from about 18 mm to about 40 mm. Thus, both the top and bottom walls 12, 14 preferably have a length that ranges from about 18 mm to about 40 mm and a width ranging from about 10 mm to about 12 mm, and each of the lateral walls 16 have a length that ranges from about 18 mm to about 40 mm and a height ranging from about 8 mm to about 18 mm. When fully constructed, the substantially rectangular or square shaped cages have a flat profile such that they are longer and wider than they are tall, making the largest points of contact of the cage with the target vertebrae the exterior surface of the top wall 12 and the exterior surface of the bottom wall 14. The lateral walls 16 may also make contact with the target vertebrae, though it is preferable that the major points of contact with the target vertebrae will occur via the exterior surface of the top wall 12 and the exterior surface of the bottom wall 14.

Both the top wall 12 and the bottom wall 14 have at least one opening or fenestration 20, and preferably a plurality of openings or fenestrations 20, that provide a location, or locations, where fusion-promoting material that is placed or stored inside of the hollow interior may freely move from the hollow interior to the exterior of the cage 10. The at least one opening or fenestration 20, thus allows for fluid communication between the hollow interior of the cage 10 and the outside of the cage 10. The purpose of these openings or fenestrations 20 is to provide a means by which a portion of a material (e.g. FPM or bone grafting material) that is placed inside of the hollow interior of the cage 10 prior to implantation may escape from the hollow interior and make contact with the target vertebrae at the top of the cage 12 and at the bottom of the cage 14, while retaining a portion of the material inside of the hollow interior. In this way, the material makes contact with one of the target vertebrae at the exterior surface of the top wall 12 of the cage and makes contact with another of the target vertebrae at the exterior surface of the bottom wall of the cage 14, while at the same time there is sufficient material remaining inside of the hollow interior so as to promote the fusion of the target vertebrae by the generation of bony or osseous tissue through and around the fusion cage 10.

Additionally, at least one of the exterior surface of the top wall or the exterior surface of the bottom wall has at least one ridge 22 located on it, which is configured to hold a shield or veneer 24 in place along the exterior surface of the particular wall. Preferably, the ridge is present on at least two of the edges of the exterior surface such that the shield or veneer 24 is held in place by the ridge 22 along at least two, and in some embodiments, three sides of the exterior surface of the wall in question. As shown in FIGS. 1 and 2, for example, the external surface of the cage 10 has a plurality of ridges 22 located on thereon and there are two ridges 22 along two opposing edges of the external surface of the top wall 12 such that the veneer 24 is held in place.

In some embodiments, as illustrated in FIGS. 1 and 2, it is preferable for at least one ridge 22 to be located along the exterior surfaces of both the top wall 12 and the bottom wall 14 so that a first veneer 24 may be held in place by at least one ridge 22 along the external surface of the top wall 12 and a second veneer 24 may be held in place by at least one ridge 22 along the external surface of the bottom wall 14. As mentioned above, the top and bottom walls 12, 14 have the at least one, and preferably a plurality of, openings or fenestrations 20 through them that allow for the movement of material out of the hollow interior of the fusion cage 10 and toward the target vertebrae. Therefore, when a first veneer 24 is held in place by at least one ridge 22 along the exterior surface of the top wall 12 and a second veneer 24 is held in place by at least one ridge 22 along the exterior surface of the bottom wall 14, the first veneer 24 and the second veneer 24 at least partially block or cover, and preferably completely block or cover, the at least one opening or fenestration 20 located along and through the top and bottom walls 12, 14. When the at least one opening or fenestration 20 is covered or blocked in this manner, the veneers 24 serve to prevent the movement of material, such as FPM or bone grafting material, from the hollow interior to the exterior of the cage 10. The veneers 24 thus serve to prevent or block the fluid communication that would otherwise exist between the hollow interior and the exterior of the cage 10. Therefore, when either the first and/or second veneer 24 is removed from contact with the ridges 22 located along the external surfaces of the top wall 12 and the bottom wall 14, fluid communication is restored between the hollow interior and the exterior of the cage 10 and the material may move from the hollow interior through the at least one opening or fenestration 20 to the outside of the cage 10.

It is also preferable for the at least one ridge 22 to be configured to hold or retain the veneer(s) 24 in place along the external surface in question sufficiently tightly so as to prevent the movement of material from the hollow interior to the outside of the cage 10. It is thus another aspect of the present disclosure for the at least one ridge 22 to hold the veneer(s) 24 in place along, and in direct contact with, the external surface of the top and/or bottom wall 12, 14 so that the veneer(s) 24 lies flat, without any wrinkles or gaps, and such that the veneer(s) 24 is held in contact with the external surface so as to prevent the movement of a substantial amount of a free flowing fluid, such as water, from the hollow interior to the outside of the cage 10, and to completely prevent the movement of a more viscous material, such as a FPM or bone grafting material, from the hollow interior to the outside of the cage 10. It is also an aspect of the present disclosure for the at least one ridge 22 to be configured to allow the veneer(s) 24 to be freely removable from the external surface of the top and/or bottom wall 12, 14 and replaceable onto the external surface of the top and/or bottom wall 12, 14 numerous times with no loss of function whatsoever. It is therefore preferable for the at least one ridge 22 to be configured so as to allow the veneer(s) 24 to move into and out of place along the external surface a plurality of times and to still hold the veneer(s) 24 in place sufficiently snugly to at least substantially prevent the movement of a viscous material from the hollow interior to the outside of the cage 10. By way of example only, the veneer(s) 24 are configured to slideably engage the cage 10 by way of the at least one ridge 22.

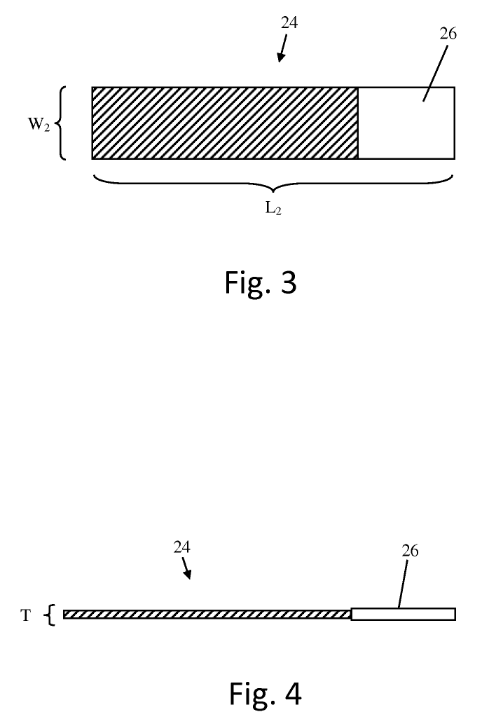

The veneer 24 may be configured to be used with any number of cage configurations, shapes and sizes. For example, the veneer 24 may be of a generally rectangular or square configuration and thus operable with the rectangular or square embodiments of the cage 10 of the present disclosure described above. Additionally, the veneer 24 may be shaped in a cylindrical, tubular, conical, circular, arced or rounded manner so as to be operable with the cylindrical or rounded embodiments of the cage 40 of the present disclosure described below. For purposes of the present disclosure, the veneer 24 may be of any shape and/or size desired including, without limitation, square, rectangular, triangular, round, circular, tubular, cylindrical, polygonal, conical, and any other shape that may be of use with an implantable intervertebral fusion cage 10 of the present disclosure. With specific reference now to FIGS. 3 and 4, an example of a veneer 24 is shown with a tab 26 according to one embodiment of the present disclosure. The veneer 24 preferably includes a projection or tab 26 located at one end that projects beyond the outer perimeter of the cage 10 and which serves to facilitate removal of the veneer 24 after implantation of the cage 10. For example, a user may grasp the projection or tab 26 at the desired time of removal and pull it in order to remove the veneer 24 from being in contact with the ridge(s) 22. Optionally, the veneer 24 may also include an opening or hole (not shown) through which a line or thread may be tied, or a surgical instrument may be inserted, in order to facilitate removal of the veneer 24 from the cage 10. FIG. 3 shows a top aspect view of the veneer 24 with the associated tab 26. A person having skill in the art will appreciate that the tab 26 may be of a material and dimensions (thickness, width, and length) different than that of the veneer 24. The veneer 24 is shown to have a width W₂ and a length L₂. FIG. 4 shows a side aspect view of the veneer 24 with the associated tab 26. The veneer 24 is shown to have a thickness T. In one embodiment of the present disclosure, the thickness T of the veneer 24 is preferably between 0.4 mm and 0.75 mm inclusive.

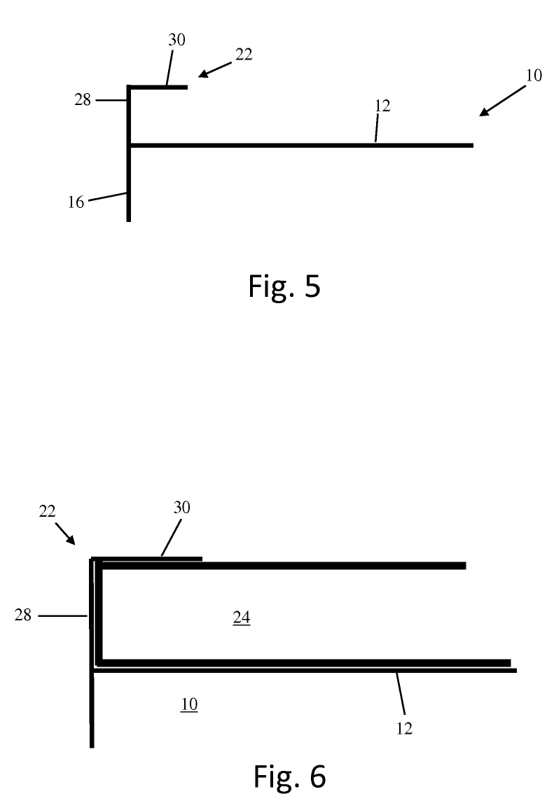

The at least one ridge 22 may be configured in any number of manners that will allow the ridge 22 to hold the veneer 24 snugly to the external surface and to allow for the free removal and replacement of the veneer 24 numerous times with no loss in function. In the embodiment described above and referring specifically to FIGS. 5 and 6, the ridge 22 is comprised of a projection of the following general L-shaped configuration:

[symbol]

In these embodiments, the bottom portion 28 of the ridge 22 projects generally perpendicularly from the top wall 12 of the cage 10, at or near the intersection between the top wall 12 and side wall 16. The upper (or overhang) portion 30 of the ridge 22 extends perpendicularly from the bottom portion 28 and projects over the top wall 12 of the fusion cage 10 in such a way so that the veneer 24 slides under the overhang 30 of the ridge 22 and is held in place by making contact with the overhang 30. The overhang portion 30 of this embodiment may vary in angle, though it is preferable that the overhang portion be at a 90 degree angle relative to the bottom portion 28.

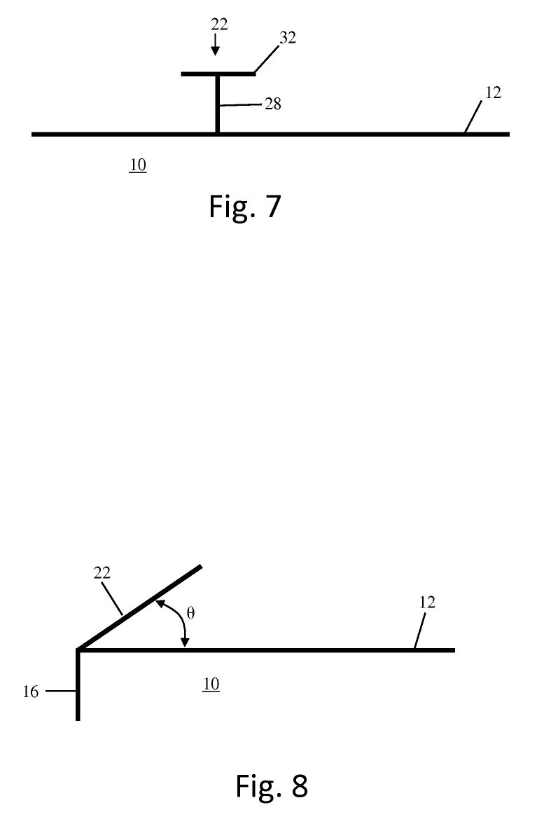

In still other embodiments and referring specifically to FIG. 7, the ridge 22 may be configured to secure a veneer 24 on two sides. The ridge 22 of these embodiments has the following general T-shaped configuration:

[symbol]

In these embodiments, the bottom portion 28 of the ridge 22 projects generally perpendicularly from the top wall 12 of the cage 10 and the upper portion (crossbar) 32, projects generally perpendicularly and outward from the bottom portion 28 in two directions, so that a veneer 24 can slide under either side of the crossbar 32 of the ridge 22 and be held in place by the crossbar 32. It is preferable for the crossbar portion 32 of these embodiments to be at a 90 degree angle relative to the bottom portion 28. This embodiment of the ridge 22 is particularly useful with cages of a round or cylindrical configuration, as described below, though they may also be used in any of the embodiments of the present disclosure.

Referring to FIG. 8, in some embodiments the ridge 22 is a single projection that extends outward from the external surface of the top wall 12 (forming an acute angle θ with the top wall 12) such that the veneer 24 slides under the ridge 22 and is held in place until such time as the veneer 24 is removed from under the ridge 22. In these embodiments, the angle θ may be configured to fit a specific veneer 24. By way of example only, the angle θ is an acute angle ranging from about zero degrees to about 45 degrees, or more preferably from about 15 degrees to about 35 degrees. In these embodiments, the veneer 24 is held in place under the ridge 22 strictly by the angle θ of the ridge 22, which makes contact with the veneer 24 along the underside of the ridge 22 and holds the veneer 24 snugly to the external surface of the top wall 12.

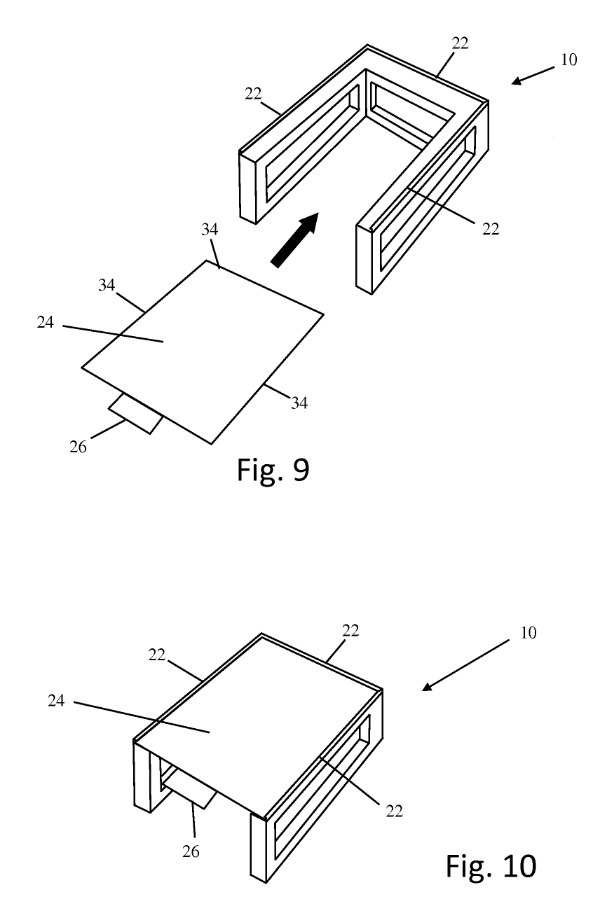

Although shown and described above as having a configuration in which a pair of ridges 22 is provided on either or both of the top and bottom walls 12, 14 in order to secure the veneer 24 in place, other configurations are possible. For example, FIGS. 9-10 illustrate an example of a fusion cage 10 in which three ridges 22 are provide on at least the top wall 12. The three ridges 22 are provided at or near the intersection of the top wall 12 with three of the side walls 16 such that the veneer 24, when inserted, is secured along three edges 34 of the veneer 24. The only edge of the generally rectangular veneer 24 (shown by way of example only) that is not secured by a ridge 22 is the trailing edge 36 containing the tab member 26. This configuration may be advantageous in that it provides security of the veneer 24 along three edges 34, including the leading edge 34, helping to ensure that the veneer 24 does not inadvertently “catch” on any anatomical structures as it is being advanced into the intervertebral space.

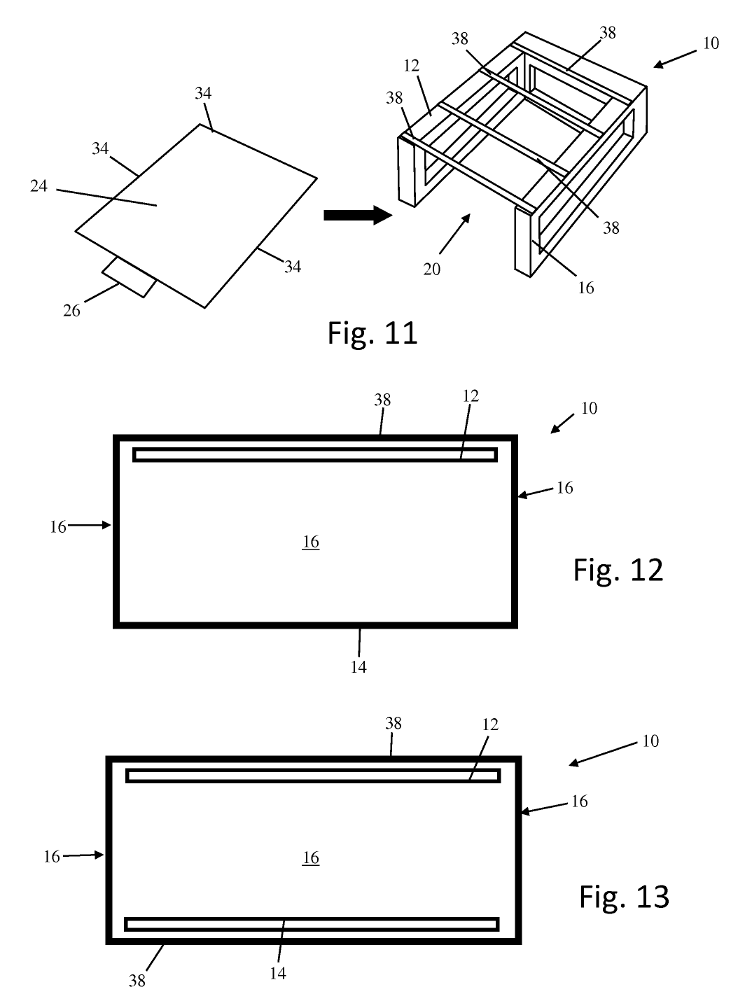

Referring to FIGS. 11-13, a further alternative example of a fusion cage 10 is a provided, with one or more belt ridges 38 extending across the top wall 12 from one of the side walls 16 to another of the side walls 16. The one or more belt ridges 38 are shown by way of example extending across the opening or fenestration 20. However, other configurations are possible. For example, one belt ridge 38 may be provided across the top wall 12 proximate to the trailing wall 16, while at least one ridge 22 is provided on the top wall 12 proximate at least one of the other side walls 16. In such a fashion, the veneer 24 would be secured along at least two edges 34, or along one edge 34 and the top surface 40 of the veneer 24. As illustrated in FIG. 13, an alternative embodiment of the fusion cage 10 may be provided having at least one belt ridge 38 extending over each of the top and bottom walls 12, 14.

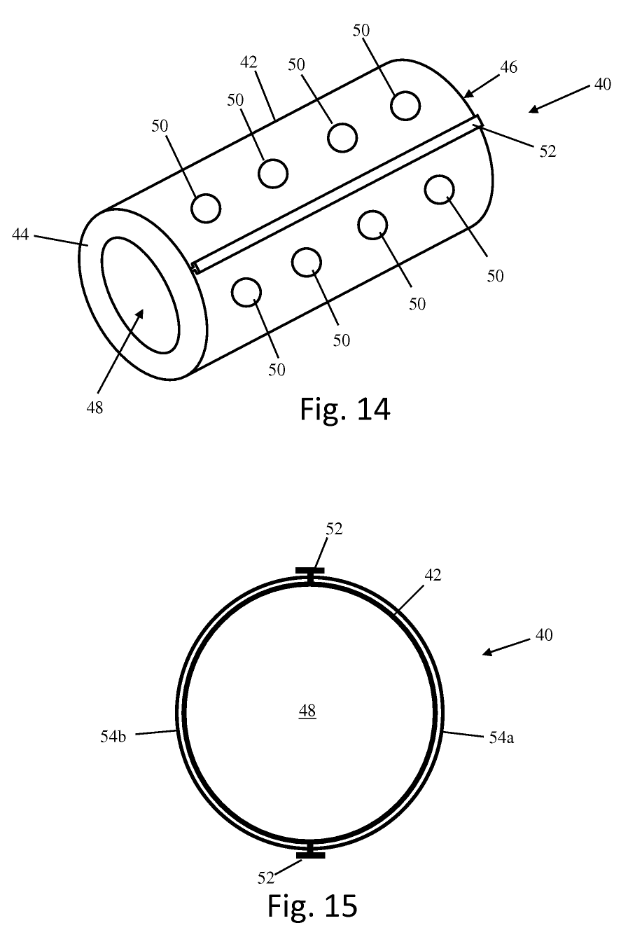

Referring to FIGS. 14 and 15, an example of a surgically implantable intervertebral fusion cage 40 having a hollow frame that is substantially tubular, cylindrical or conical in shape is provided according to one embodiment of the present invention. For the sake of simplicity, the cylindrical cage will be referred to herein as having a main wall 42 that is tubular, cylindrical, or conical in shape and/or configuration, a first end wall 44 and a second end wall 46, each end wall 44, 46 being interconnected with the main wall 42 so as to form a closed tube, cylinder, or cone in that the main wall 42 is connected to the first end wall 44 and to the second end wall 46 in such a way that the main wall 42, first end wall 44 and second end wall 46, when fully interconnected, define a hollow interior 48 to the cage 40 that is tubular, cylindrical or conical in shape and/or configuration. Preferably, the intervertebral fusion cage 40 of the present embodiment has a diameter ranging from about 8 mm to about 18 mm and a length ranging from about 18 mm to about 40 mm. Thus, both the first end wall 44 and the second end wall 46 preferably have a diameter that ranges from about 8 mm to about 18 mm and the main wall has a length that ranges from about 18 mm to about 40 mm. When fully constructed, the tubular, cylindrical or conical shaped cages have an elongated appearance such that they are longer than they are tall, making the largest points of contact of the cage with the target vertebrae two arcs along the external surface of the main wall 42, one at the top of the surgical site that makes contact with one of the target vertebrae and one at the bottom of the surgical site that makes contact with a second target vertebra. The first end wall 44 and the second end wall 46 may also make contact with the target vertebrae, though it is preferable that the major points of contact with the target vertebrae will occur via the exterior surface of the main wall 42.

The main wall 42 has at least one opening or fenestration 50, and preferably a plurality of openings or fenestrations 50, located along its length that provide a location, or locations, where material that is placed or stored inside of the hollow interior 48 may freely move from the hollow interior 48 to the exterior of the cage 40. The at least one opening or fenestration 50 thus allows for fluid communication between the hollow interior 48 of the cage 40 and the outside of the cage 40 in a similar manner as described above.

Additionally, the exterior surface of the main wall 42 has at least one ridge 52 located on it, which is configured to hold at least one shield or veneer 54 in place along the exterior surface of the main wall 42. In some embodiments, as illustrated by way of example only in FIG. 14, a single, T-shaped ridge 52 runs the entire length of the exterior surface of the main wall 42 such that a single shield or veneer 54 can be wrapped around the full exterior circumference of the main wall 42 and be held in place by making contact with both sides of the T-shaped ridge 52. As stated above, all embodiments of the ridge 52 may be used to secure one or more veneers 54 to the external surface of a cage 40 of these embodiments, though the embodiment of the ridge 54 that is particularly useful for a cage 40 of this shape and/or configuration is the T-shaped ridge 54 shown and described above. In other embodiments and with specific reference now to FIG. 15, the external surface has at least one ridge 52, and may also have a plurality of ridges 52, located thereon and there may be two ridges 52 running the length of two opposing sides of the external surface of the main wall 42 such that two veneers 54, a first veneer 54a and a second veneer 54b, are held in place along two opposing sides of the external surface of the main wall 42. In one embodiment, there are a plurality of ridges 52 holding the veneer(s) 54 in place, at least one ridge 52 running the length of the external surface of the main wall 42 as previously described and at least one ridge (not shown) located in a belt-like fashion around the entire perimeter of the external surface of the main wall 42 such that the veneer 54 slides under the at least one belt-like ridge, makes contact with the other at least one ridge 54, and is held in place accordingly.

As mentioned above, the main wall 42 has at least one, and preferably a plurality of, openings or fenestrations 50 formed therein that allow for the movement of material out of the hollow interior 48 and toward the target vertebrae. Therefore, when a veneer 54 is held in place by a ridge 52 along the exterior surface of the main wall 42, the veneer 54 at least partially blocks or covers, and preferably completely blocks or covers, the at least one opening or fenestration 50 located along and through the main wall 42. When the at least one opening or fenestration 50, and preferably the plurality of openings or fenestrations 50, are covered or blocked in this manner, the veneer 54 serves to prevent the movement of material as previously described. Additionally, when the veneer 54 is removed from contact with the ridge(s) 52 located along the external surface of the main wall 42, fluid communication is restored as previously described.

Further description of the features disclosed herein will continue in reference to the example of the interbody fusion cage 10 having ridges 22 disclosed above in relation to FIGS. 1 and 2, however the discussion that follows applies equally to the fusion cage 40 having ridges 52 disclosed above in relation to FIGS. 14-15. The ridges 22 of the present disclosure may be present along the external surface of the cage 10 in any number of manners. For example, in some embodiments the ridges 22 are cast with the cage 10 as an extension of the external surface of the top and/or bottom walls 12, 14 of the cage 10 itself, thereby making the ridge 22 a continuous physical extension of the cage 10. In other embodiments, the ridge 22 is ground out of or machined out of the exterior surface of the cage wall. In these embodiments, the ridge 22 may be ground out or machined out at the time the cage 10 is ground out or machined out of a portion of a larger material, or the ridge 22 may be ground out or machined out in advance of implantation in order to provide a customized ridge 22. In still other embodiments, the ridge 22 may be a separate piece that is attached to, or secured onto, the external surface of the cage 10 prior to implantation. The ridge 22 of these embodiments may be secured onto the external surface by any one or more of many standard means by which two structures may be operably connected together, such as with the use of adhesives, welding, bands, straps, threading, a clamp, a snap-fit assembly, a bolted or screwed connection, a push-on/turn-on self-locking fastener, a press fit, rivets, and/or other, similar means. In other embodiments of the present disclosure, the ridge 22 may be present on an internal surface of the cage 10.

The surgically implantable intervertebral fusion cage 10 of the present invention, inclusive of the ridges 22 disclosed herein, may be made of any kind of material suitable for surgical implantation that is also sufficiently rigid so as to provide the required support between the target vertebrae, such as steel or medical grade plastic. In some embodiments, the ridge 22 is an integral part of the cage 10 and the two structures are cast as a single, continuous unit. The cages 10 and the ridges 22 may be made of: surgical stainless steel of the general alloy type of iron, carbon, chromium (12-20%), molybdenum (0.2-3%), and nickel (8-12%); martensitic steel; 316L or 316LVM austenitic steel; and/or 316 surgical steel. It is desirable for the cages 10 and ridges 22 of the present invention to be quite rigid and strong in construction so as to prevent any deforming during use and/or after surgical implantation, which can cause a loss of function.

The veneers 24 of the present invention may be made of any kind of material suitable for surgical implantation that is rigid enough to completely prevent the movement of viscous materials from the hollow interior when the veneer 24 is in place along the exterior surface, but that is also sufficiently flexible so as to be readily removable and replaceable without being damaged or deformed in any way. Suitable materials include plastic, poly(tetrafluoroethene) or poly(tetrafluoroethylene), or plastic, though the veneers 24 may also be made of polyamide, polyethylene, polypropylene, polyphenylene sulfide, polyurethane, poly(tetrafluoroethylene), polyvinyl chloride, polyvinylidene fluoride or polyetheretherketone (PEEK).

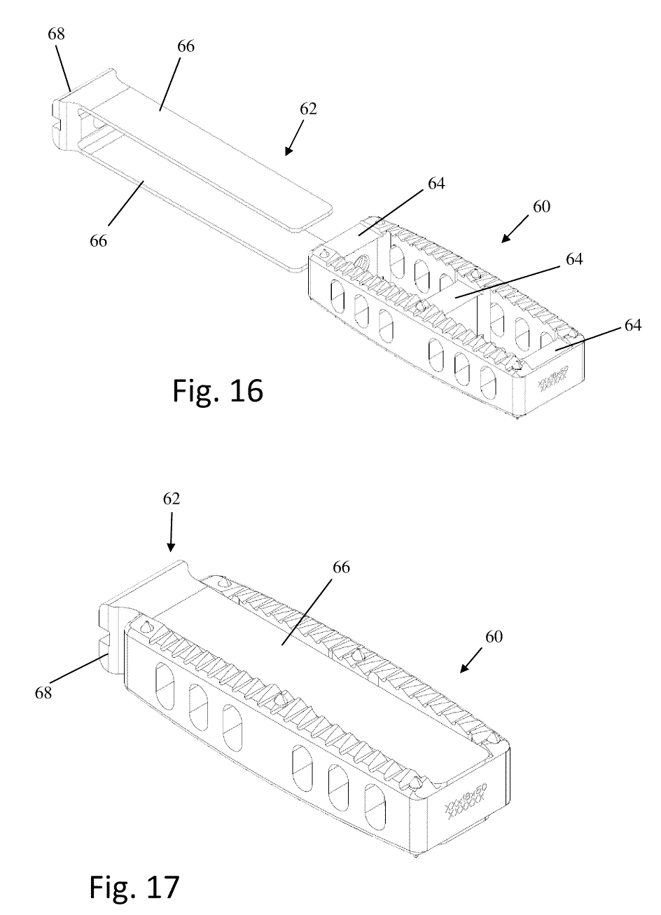

FIGS. 16 and 17 illustrate an example of a combination of a fusion cage 60 and veneer 62 according to an alternative embodiment of the present invention. The fusion cage 60 is similar to that described above in relation to FIG. 1 and further, however one difference between the fusion cage 10 and the fusion cage 60 is the absence of a ridge on the fusion cage 60 having the structure of a projection to securely couple the veneer 62 and the cage 60. Rather, as will be described in further detail below, the fusion cage 60 is provided with recesses 64 formed within the top and bottom surfaces, the recesses being configured to receive the tangs of the veneer. The veneer 62 is also similar to the veneer 24 as described in relation to FIG. 3 and further, however the veneer 62 of the current embodiment is described as having two tangs 66 projecting in a generally parallel manner from a body 68. As will be described in further detail below, the body 68 is configured to be attached to any number of insertion instruments, enabling ease of operation of the combination of the fusion cage 60 and veneer 62 described herein.

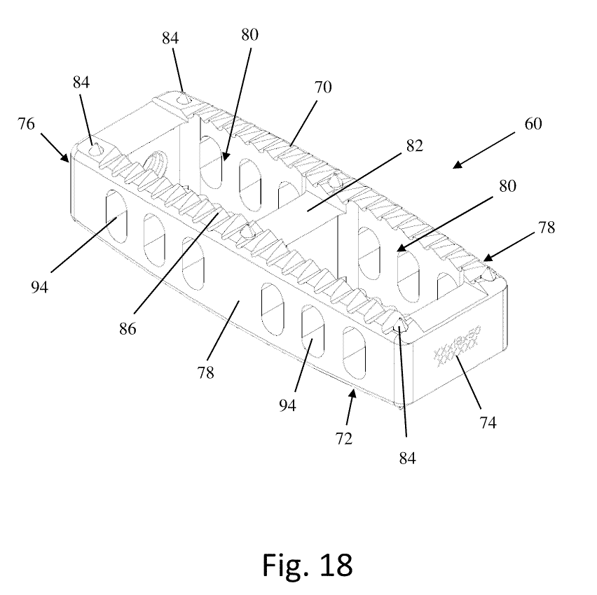

Referring to FIGS. 18-22, the fusion cage 60 described herein has a generally rectangular cross-sectional shape, and includes a top surface 70, a bottom surface 72, a leading wall 74, a trailing wall 76, and a pair of elongated side walls 78, the configuration of walls defining a hollow interior therebetween. The top and bottom surfaces 70, 72 are interrupted by at least one, and in the example shown, two openings or fenestrations 80 extending through the top and bottom surfaces 70, 72 to provide communication between the hollow interior of the cage 60 and the exterior environment of the cage 60. Further, in the example shown in FIG. 18, the fusion cage 60 further includes a middle portion 82 that provides structural support to the cage 60 and effectively divides the hollow interior into two chambers.

The top and bottom surfaces 70, 72 are provided with a plurality of anti-migration features to help prevent the cage 60 from moving within the intervertebral disk space after implantation. By way of example only, such anti-migration features include a plurality of spikes 84, and surfaces ridges 86.

The leading end 74 is shown by way of example as being generally planar in shape, however any shape and configuration may be provided without departing from the scope of the present invention, including rounded, sloped, bullet-shaped, etc.

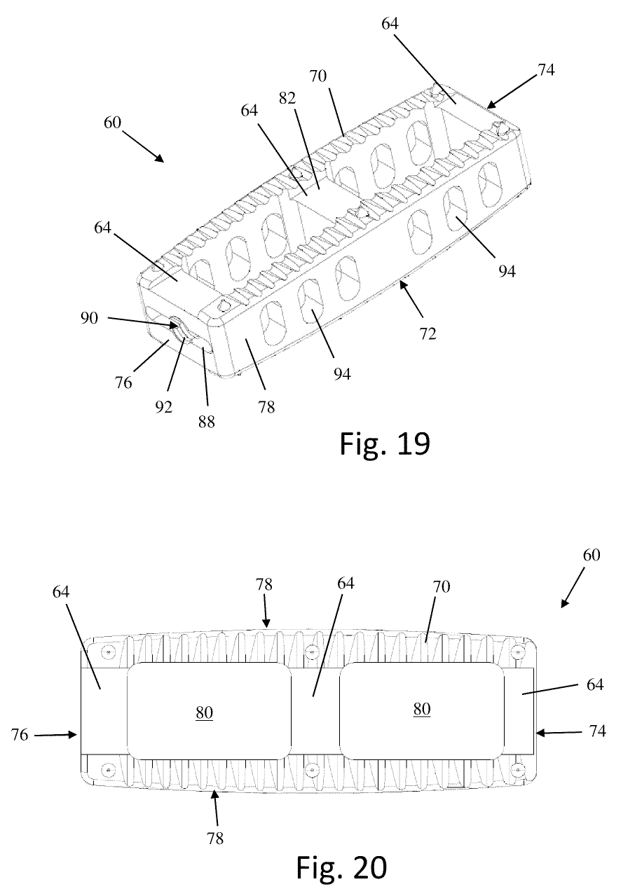

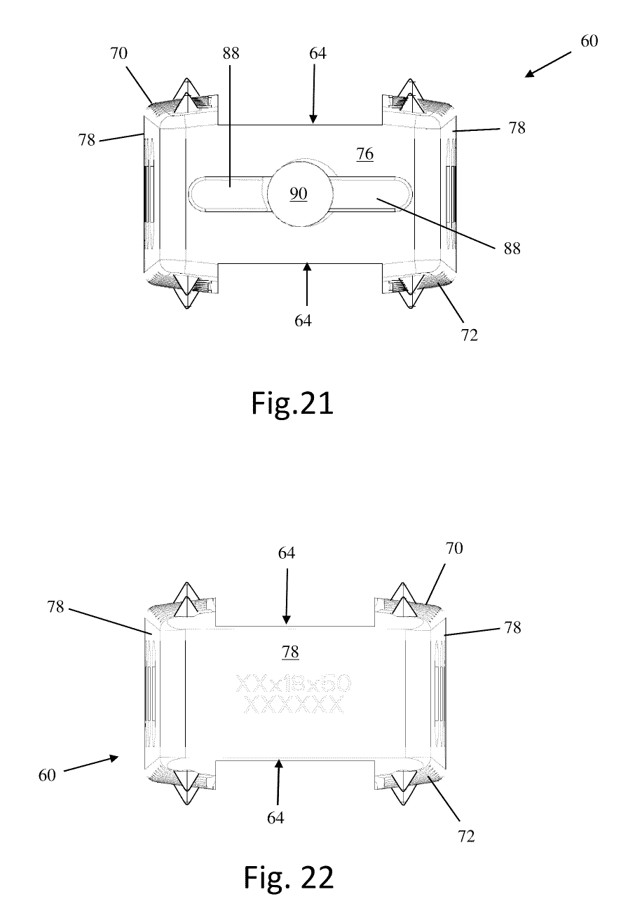

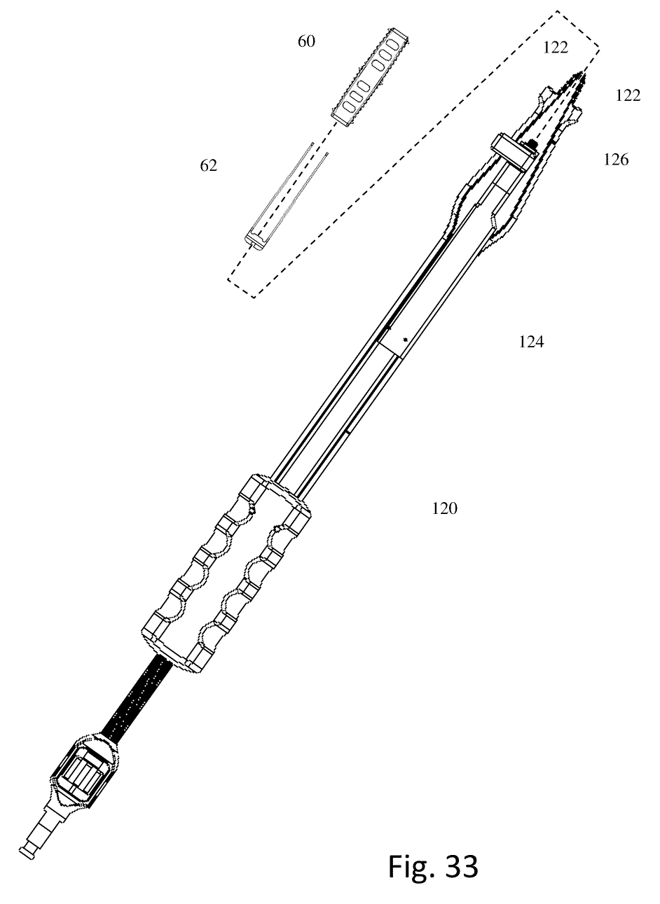

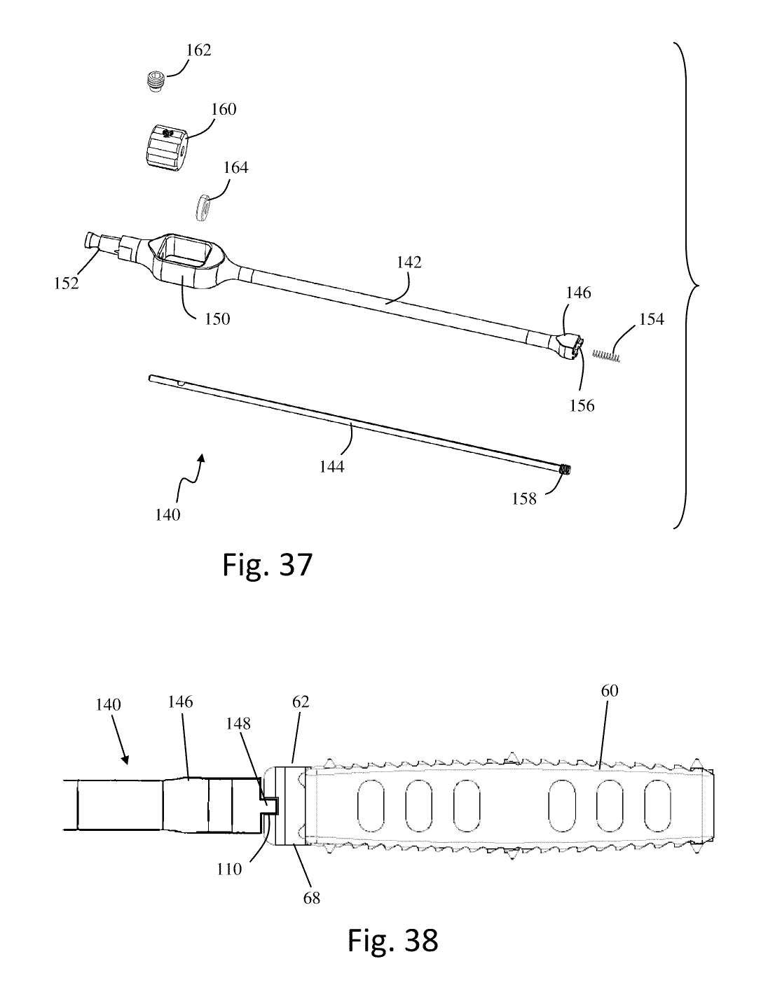

As seen most clearly in FIGS. 19 and 21, the trailing end 76 includes an elongated recess 88 formed within the surface of the trailing end 76 and an aperture 90 extending from the external surface of the trailing end 76 to the hollow interior of the fusion cage 60. The elongated recess 88 is configured and dimensioned to receive an anti-rotation feature of an implant inserter, several examples of which are described in further detail with reference to FIGS. 33-37 below. The aperture 90 is generally cylindrical in shape, and is provided by way of example only with a threaded interior surface 92. The threaded surface 92 is configured to engage with a threaded member on the various examples of the inserters 120, 140, as will be described in further detail below.

The top and bottom surfaces 70, 72 are each provided with a plurality of recesses 64. The recesses extend substantially across the top and bottom surfaces and are approximately equal to the width of the interior chamber of the cage 60. The recesses 64 have generally planar surfaces which are adapted to slideably receive the tangs 66 of the veneer 62.

Optionally, the side walls 78 may be provided with one or more apertures 94. Apertures 94 are generally small enough to limit the egress of liquids with a relatively high viscosity, and so do not generally pose a problem related to leakage of FPMs.

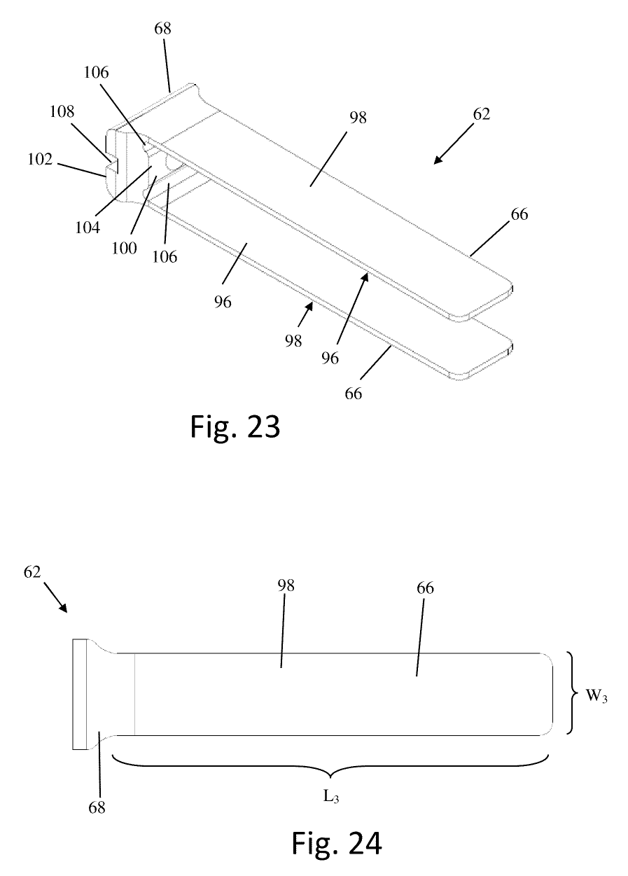

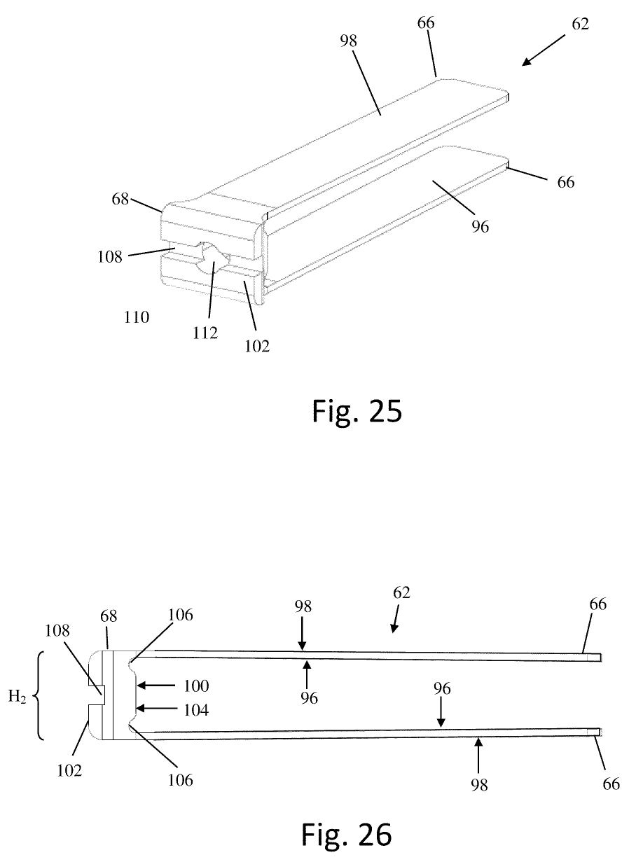

Referring to FIGS. 23-26, the veneer 62 described herein by way of example only includes two tangs 66 projecting in a generally parallel manner from a body 68. By way of example only, the tangs 66 are elongated, generally planar members that have a generally rectangular cross-section. Each tang 66 has a first surface 96 and a second surface 98, with the first and second surfaces 96, 98 being on opposite sides of the tang 66. The first surfaces 96 of the tangs 66 are oriented such that they face one another, and thus may be synonymously referred to as “inside surfaces 96” without departing from the present invention. The second surfaces 98 are oriented such that they face away from one another and are on the exterior of the veneer 62, and thus may be synonymously referred to as “outside surfaces 98” without departing from the present invention. The inside surfaces 96 are generally planar and are dimensioned to slideably engage the recesses 64 provided on the fusion cage 60 described above. The outside surfaces 98 may be generally planar or have a convex curvature.

The tangs 66 have a length dimension L₃ and a height dimension H₃. The length dimension L₃ corresponds directly to the length dimension of the fusion cage 60. By way of example only, the length dimension L₃ is within a range of about 18 mm to about 40 mm. The width dimension W₃ is within a range of about 10 mm to about 12 mm. These dimension ranges are provided by way of example only, and implants of different size and shape may be provided outside of these ranges depending upon the particular needs of the user.

The tangs 66 are connected to a body 68 such that the veneer 62 has an overall generally U-shaped configuration. The body 68 has a height dimension H₂ that defines the distance that the tangs are spaced apart. This distance may be in the range of about 6 mm to about 18 mm. The body 68 further includes a leading side 100 and a trailing side 102. The leading side 100 is on the “inside” of the veneer 62 (e.g. such that the leading side 100 faces the inside surfaces 96 of the tangs 66), and includes a generally planar surface 104 for abutting the training end 76 of the fusion cage 60. Upon either side of the abutment surface 104, and between the abutment surface 104 and the tangs 66, is a generally concave recess 106. The recess 106 is provided to give the tangs 66 the ability to flex without creating undue stress on the veneer 62.

The trailing side 102 includes an elongated recess 108 and an aperture 110 formed therein. The elongated recess 102 is formed within the outer surface of the is configured and dimensioned to receive an anti-rotation feature of an implant inserter, several examples of which are described in further detail with reference to FIGS. 33-37 below. The aperture 110 is generally cylindrical in shape, and is provided by way of example only with an interior surface 112 that may be either smooth or threaded depending upon the particular needs of the user. The aperture 110 extends completely through the body 68, from the trailing side 102 to the leading side 100. The aperture 110 is positioned such that upon coupling of the fusion cage 60 and the veneer 62, the aperture 110 is coaxially aligned with the aperture 90 of the fusion cage 60. The interior surface 112 is configured to engage with a threaded member on the various examples of the inserters 120, 140, as will be described in further detail below.

The tangs 66 are described above as being generally parallel to one another, however one embodiment of the veneer 62 includes tangs 66 that are biased in a slightly convergent relationship to one another, as shown in FIG. 26. This slight convergence causes the tangs 66 to exert a force or pressure upon the fusion cage 60 after coupling of the cage 60 and the veneer 62, which serves as one securement feature in lieu of securement ridges in this embodiment. The force or pressure helps ensure that the tangs 66 do not separate during insertion, which would allow premature egress of the FPM that is inserted in the hollow interior of the cage 60.

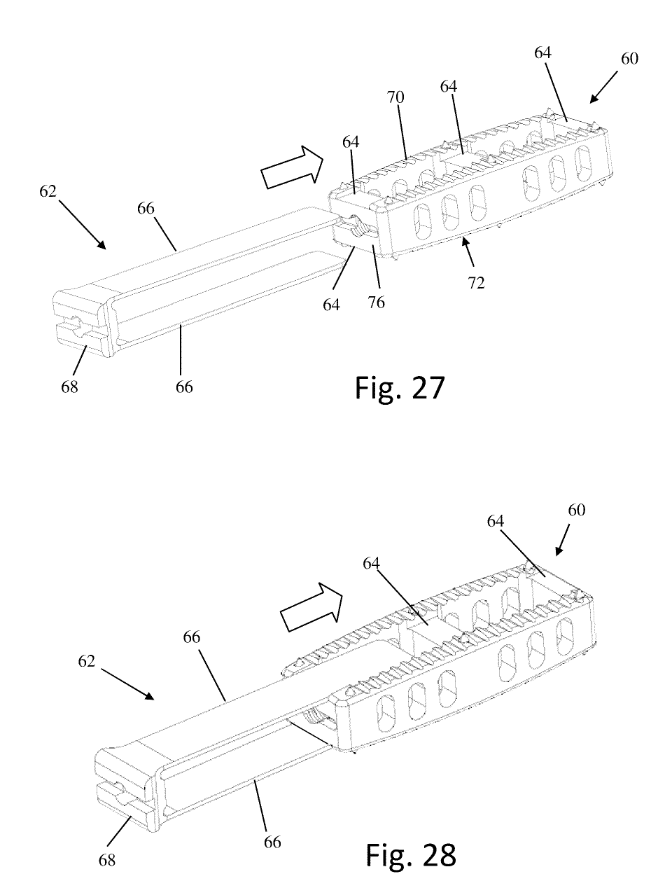

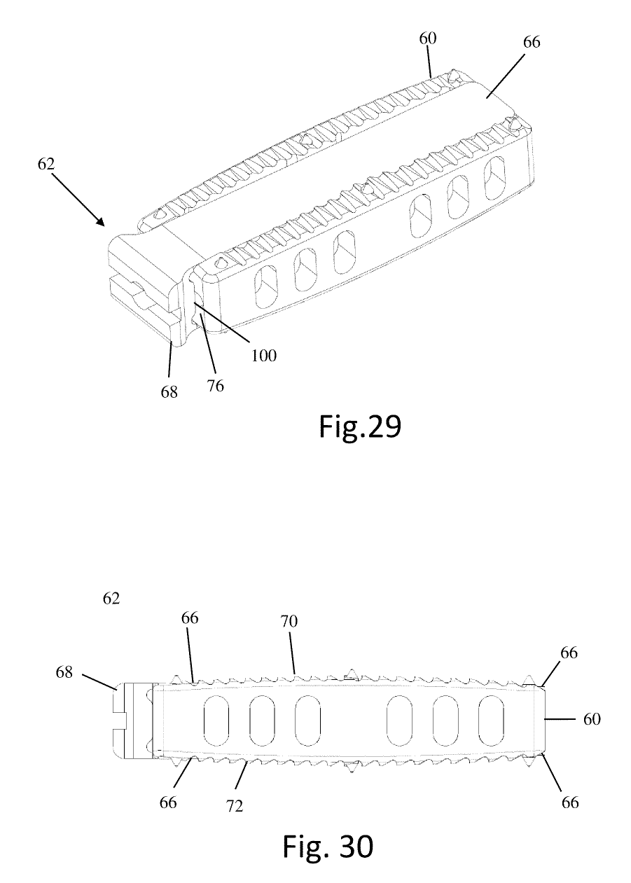

Referring to FIGS. 27-29, a process of coupling the fusion cage 60 and the veneer 62 is shown according to one embodiment of the present invention. Referring to FIG. 27, the fusion cage 60 and veneer 62 are aligned such that the tangs 66 approach the trailing end 76 of the fusion cage 60 in line with the recesses 64 formed in the top and bottom surfaces 70, 72. The veneer 62 is then advanced on to the cage 60 toward the leading end 74 of the cage 10. The veneer 62 is advanced until the leading side 100 of the body 68 comes into flush contact with the trailing end 76 of the fusion cage 60, as shown in FIG. 29. At this point, the apertures 90, 110 of the fusion cage 60 and body 68, respectively, will be in coaxial alignment and ready to receive a portion of an inserter 120, 140, as will be described in further detail below.

FIGS. 30-32 illustrate the fusion cage 60 and veneer 62 in a fully coupled association. As evident by way of example in FIG. 30, the veneer 62 and fusion cage 60 interact in such a fashion so that there is no additional profile added to the construct. In other words, the tangs 66 fit with the recesses 64 of the fusion cage 60 such that no portion of the tangs 66 extend beyond the exterior limits of the top and bottom surfaces 70, 72. This ensures minimizes any potential for negative impact on the fusion surgery by the veneer 62.

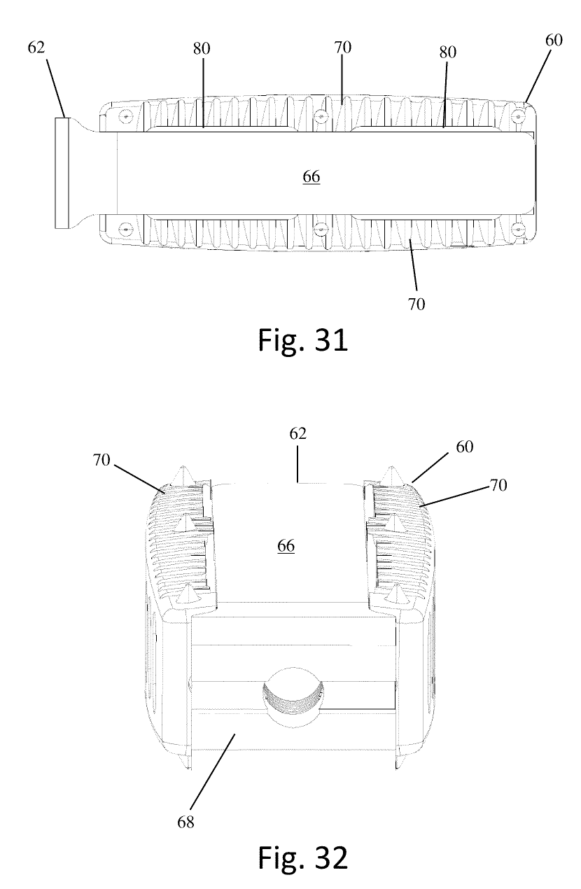

The example shown in FIG. 31 indicates that the tangs 66 do not fully cover the apertures or fenestrations 80. This is one example of how the cage 60 and veneer 62 may be sized to interact, and does not pose any problem for highly viscous FPMs. In other examples, the tangs 66 may be provided with sufficient width to completely block the apertures 80, depending upon the particular needs of the user.

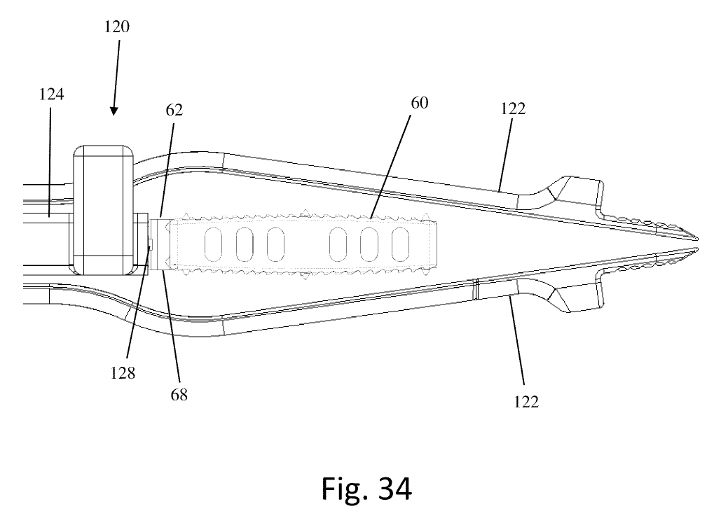

FIGS. 33 and 34 illustrate one example of an inserter 120 for use in inserting the fusion cage 60 and veneer 62 combination into an intervertebral space. The inserter 120 is of the type generally shown and described in commonly owned and co-pending U.S. Application Ser. No. 61/028,886, filed Feb. 14, 2008 and entitled “Spinal Distraction and Implantation Assembly and Related Methods,” and U.S. Application Ser. No. 61/105,384, filed Oct. 14, 2008 and entitled “Spinal Distraction and Implantation Assembly and Related Methods,” the complete disclosures of which are hereby incorporated by reference into this application as if set forth fully herein.

Generally, the inserter 120 includes a pair of shim members 122 that are configured to be inserted into an intervertebral space, and a pusher 124 disposed in between the shim members 122. The pusher member 124 includes a tip 126 that by way of example only is threaded. The threaded tip 126 is generally cylindrical in shape, and configured to engage an implant for insertion into the intervertebral space. In this instance, the tip 126 is sized such that it may be advanced through aperture 110 of the veneer 62 and threadedly engaged with the threadform 92 of the aperture 90 of the fusion cage 60.

As shown in FIG. 34, the pusher 124 further includes an elongated stabilizer 128 to assist in prohibiting the rotation of an implant while being threadedly engaged to the tip 126 and/or inserted into the disc space. In this instance, the elongated stabilizer 128 is sized to flushly mate with the elongated recess 108 of the veneer 62. This mating ensures the fusion cage 60 and veneer 62 combination will remain rotationally stabilized throughout the insertion process.

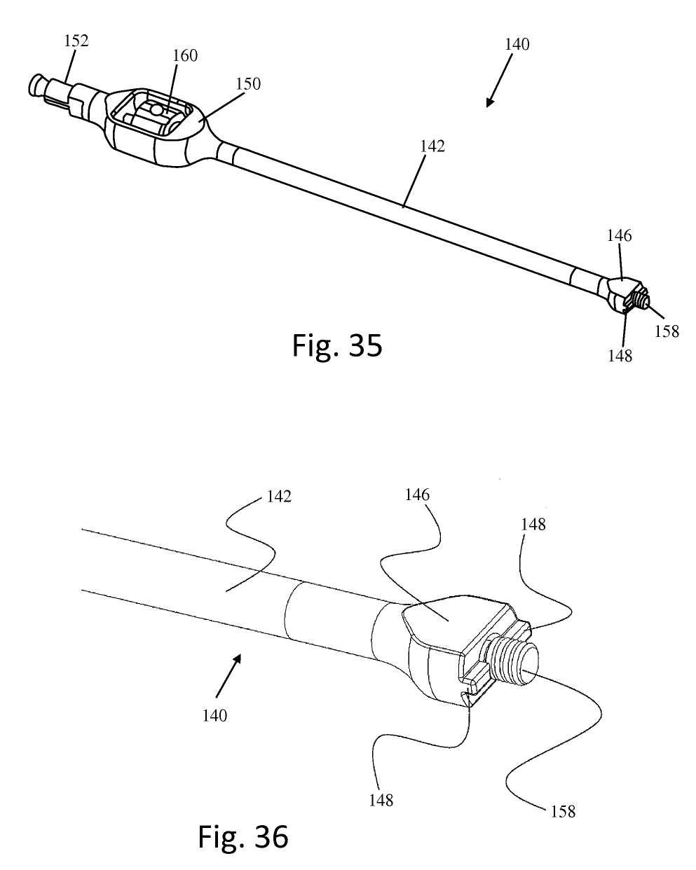

FIGS. 35-38 illustrate an example of an inserter 140 for use in inserting the fusion cage 60 and veneer 62 combination into an intervertebral space, according to an alternative embodiment of the present invention. The inserter 140 is of the type generally shown and described in commonly owned and co-pending U.S. application Ser. No. 11/093,409, filed Mar. 29, 2005 and entitled “Systems and Methods for Spinal Fusion,” the entire contents of which are hereby incorporated by reference into this disclosure as if set forth fully herein.

Generally, the inserter 140 includes an elongate tubular element 142 and an inserter shaft 144. The elongate tubular element 142 is constructed with a distal head 146 at its distal end, distal head ridges 148 on the distal end of the distal head 146, a thumbwheel housing 150 at its proximal end and a handle 152 at its proximal end. The elongate tubular element 142 is generally cylindrical and of a length sufficient to allow the device to span from the surgical target site to a location sufficiently outside the patient's body so the handle 152 and thumbwheel housing 150 can be easily accessed by a clinician or a complimentary controlling device.

The elongate tubular element 142 is dimensioned to receive a spring 154 and the proximal end of the inserter shaft 144 into the inner bore 156 of the elongate tubular element 142. The inserter shaft 144 is dimensioned such that the threaded connector 158 at the distal end of the inserter shaft 144 just protrudes past the distal head ridges 148 to allow engagement with the aperture 90 of the spinal fusion implant 60. It should be appreciated by one skilled in the art that such a construction allows the inserter shaft 144 to be able to rotate freely within the elongate tubular element 142 while stabilized by a spring 154 to reduce any slidable play in the inserter 140.

The handle 152 is generally disposed at the proximal end of the inserter 140. The handle 152 is fixed to the thumbwheel housing 150 allowing easy handling by the clinician. Because the handle 152 is fixed, the clinician has easy access to the thumbwheel 160 and can stably turn the thumbwheel 160 relative to the thumbwheel housing 150. Additionally, the relative orientation of the thumbwheel housing 150 to the handle 152 orients the clinician with respect to the distal head 146 and distal head ridge 148. By way of example, the thumbwheel housing 150 holds a thumbwheel 160, a set screw 162, and a spacer 164. The inserter shaft 144 is attached to the thumbwheel 160 and is freely rotatable with low friction due to the spacer 164. One skilled in the art can appreciate myriad methods of assembling a housing similar to the above described.

FIG. 38 details the distal head 146 of the inserter 140 coupled to the spinal fusion implant 60 and the veneer 62. The distal head ridges 148 are dimensioned to fit slidably into the recess 110 of the veneer 62 with low friction to allow accurate engagement of the threaded connector 158 to the threaded aperture 90 of the spinal fusion implant 60. It is through these interactions that the veneer 62 and the spinal fusion implant 60 are secured to the inserter 140.

In accordance with at least some aspects of at least one embodiment of the present invention, a method of surgically implanting an intervertebral fusion cage 10 into a patient is provided. The method comprises first obtaining a surgically implantable intervertebral fusion cage 10 having at least one wall (for example top wall 12), at least one ridge 22 located on an exterior surface of the at least one wall 12, and at least one removable shield or veneer 24. The at least one wall 12 defines a hollow interior to the cage 10 and has at least one opening or fenestration 20 in it that allows for fluid communication between the hollow interior and an exterior of the cage 10. The ridge 22 is operable to hold the at least one veneer 24 in contact with the exterior surface and is configured to allow the at least one veneer 24 to be freely removed from, and replaced onto, the exterior surface of the wall 12.

The next step of the method is to prepare the cage 10 for surgical implantation by filling the hollow interior with a material capable of fusing two bony structures, preferably two vertebrae, and more preferably two adjacent vertebrae, together. For example this material may be an orthopedic matrix containing additional fusion-promoting material (FPM), for example including but not limited to calcium hydroxyapatite, bone morphogenic protein (BMP), demineralized bone matrix, collagen bone graft matrix (e.g. Formagraft®) and stem cell material (e.g. Osteocel®) or other fusion-promoting substance placed within the spaces of the implant. The next step is contacting the at least one veneer 24 with the exterior surface of the cage 10 by positioning the veneer 24 under at least a portion of the at least one ridge 22 such that the at least one veneer 24 is contacted with and held in place along the exterior surface of the top wall 12 by the at least one ridge 22 by way of such contact. Preferably, when the at least one veneer 24 is contacted with the at least one ridge 22, the veneer 24 at least partially covers or blocks the at least one opening or fenestration 20, preventing fluid communication between the hollow interior and the exterior of the cage 10, and preventing the material from leaving the hollow interior.

After the cage 10 has been prepared for implantation, the next step of the method is to locate an appropriate surgical site in a patient for implantation of the cage 10. The surgical site may be an intervertebral location, including the space typically filled by an intervertebral disc, but may also be any location in a patient where two bony structures are to be fused together. The next step in the method is to create a surgical opening and operative corridor in the patient that will accommodate the cage 10. This opening may be made dorsally, ventrally, laterally or at any other location along the patient that is medically efficacious to grant the user access to the surgical site. Once the opening is created, the next step in the method is to surgically implant the cage 10 into the patient and then position it in the surgical site between the desired bony structures, and preferably between the two adjacent vertebrae of interest, in such a way that will serve to utilize the material in connection with the patient's own systems to promote the fusion of the two bony structures by way of, and though, the cage 10. Thereafter, the at least one veneer 24 is removed from contact with the ridge and the exterior surface of the cage 10, thereby restoring fluid communication between the hollow interior and the exterior of the cage 10 and allowing the material to move from the hollow interior to the exterior of the cage 10. Once these tasks are completed, the method is concluded by closing the surgical opening in the patient.

While this invention has been described in terms of a best mode for achieving this invention's objectives, it is understood by those skilled in the art that variations may be accomplished in view of these teachings without deviating from the spirit or scope of the invention. Moreover, the various embodiments disclosed herein are provided by way of example only, and the specific features described in relation to the specific embodiments are not limited to those specific embodiments, but rather may be provided interchangeably and in combination with any of the various features disclosed herein without departing from the scope of the present invention.

What is claimed is:

1. An implantable intervertebral fusion cage comprising:
a substantially rectangular frame having a top wall, a bottom wall and four lateral walls, each lateral wall being interconnected with the top wall at a top edge of the lateral wall, each lateral wall being interconnected with the bottom wall at a bottom edge of the lateral wall, wherein the top wall, bottom wall and four lateral walls define a hollow interior and an exterior of the cage, and the top wall and the bottom wall each have at least one opening allowing for fluid communication between the hollow interior and an exterior of the cage;
at least one ridge located on an exterior surface of the top wall; and
at least one generally planar veneer sized and configured to at least partially cover the at least one opening in the top wall;
wherein the at least one ridge located on an exterior surface of the top wall is configured to reversibly hold the at least one veneer in contact with the exterior surface of the top wall so as to at least partially cover the at least one opening in the top wall.

2. The intervertebral fusion cage of claim 1, further comprising:
at least one ridge located on an exterior surface of the bottom wall, wherein the at least one ridge located on an exterior surface of the bottom wall is configured to reversibly hold the at least one veneer in contact with the exterior surface of the bottom wall so as to at least partially cover the at least one opening in the bottom wall.

3. The intervertebral cage of claim 1, wherein the at least one ridge located on an exterior surface of the top wall is configured to allow the at least one veneer to be removed from contact with the exterior surface of the top wall.

4. The intervertebral cage of claim 2, wherein the at least one ridge located on an exterior surface of the bottom wall is configured to allow the at least one veneer to be removed from contact with the exterior surface of the bottom wall.

5. The intervertebral cage of claim 1, wherein the frame is substantially a shape selected from a group consisting of a square, a trapezoid, a parallelogram and a triangle.

6. A system for performing spinal fusion, comprising:
a intervertebral fusion cage having a substantially rectangular frame having a top wall, a bottom wall, a leading end, a trailing end, and a pair of lateral walls, the intervertebral fusion cage having a hollow interior and an exterior, and the top wall and the bottom wall each have at least one opening allowing for fluid communication between the hollow interior and an exterior of the cage, at least one recess located on an exterior surface of the top wall, and at least one recess located on an exterior surface of the bottom wall;
a fusion promoting material configured for placement within the hollow interior of the fusion cage;
a veneer having first and second elongated tangs joined at one end by a body, the elongated tangs having a generally rectangular planar shape;
wherein said first tang is configured to slidingly engage the at least one recess located on an exterior surface of the top wall, and said second tang is configured to slidingly engage the at least one recess located on an exterior surface of the bottom wall, said veneer configured to reversibly hold the intervertebral fusion cage in contact with the tangs so as to at least partially cover the at least one opening in the top wall and the at least one opening in the bottom wall.

7. The system of claim 6, wherein said tangs extend from said base in a parallel manner.

8. The system of claim 6, wherein said tangs extend from said base in a convergent manner.

9. The system of claim 6, further comprising an inserter configured to simultaneously retain the intervertebral fusion cage and the veneer in a rotationally secure state.

10. The system of claim 9, wherein said intervertebral fusion cage includes a first aperture formed in said trailing end, said first aperture configured to mate with said inserter.

11. The system of claim 10, wherein said first aperture is threaded.

12. The system of claim 11, wherein said body of said veneer includes a leading side and a trailing side.

13. The system of claim 12, wherein said body of said veneer includes a second aperture extending through said body from said leading side to said trailing side.

14. The system of claim 12, wherein said first and second apertures are coaxially aligned with one another.

15. The system of claim 6, wherein the fusion-promoting material is at least one of calcium hydroxyapatite, bone morphogenic protein, demineralized bone matrix, collagen bone graft matrix, and stem cell material.

16. A method for surgically implanting an intervertebral fusion cage into a patient, comprising:
providing a surgically implantable intervertebral fusion cage having a top wall, a bottom wall, a leading end, a trailing end, and a pair of lateral walls, the intervertebral fusion cage having a hollow interior and an exterior, and the top wall and the bottom wall each have at least one opening allowing for fluid communication between the hollow interior and an exterior of the cage, at least one recess located on an exterior surface of the top wall, and at least one recess located on an exterior surface of the bottom wall;
filling the hollow interior with a material capable of promoting the fusion of two bony structures;
providing a veneer having first and second elongated tangs joined at one end by a body, the elongated tangs having a generally rectangular planar shape, the first tang configured to slidingly engage the at least one recess located on the top wall and the second tang configured to slidingly engage the at least one recess located on the bottom wall;
positioning the fusion cage within the veneer such that the first tang at least partially covers the at least one opening in the top wall and the second tang at least partially covers the at least one opening in the bottom wall;
locating an appropriate surgical site in the patient for implantation of the cage;
creating a surgical opening in the patient to accommodate access to the appropriate surgical site and implantation of the cage;
moving the cage into the surgical opening and into the surgical site;
removing the veneer from the exterior surface of the cage; and
closing the surgical opening.

17. The method of claim 16, further comprising the step of
simultaneously coupling the intervertebral fusion cage and the veneer to an inserter in a rotationally secure state.

18. The method of claim 17, wherein said intervertebral fusion cage includes a first aperture formed in said trailing end, said first aperture configured to mate with said inserter.

19. The method of claim 18, wherein said body of said veneer includes a leading side and a trailing side, and a second aperture extending through said body from said leading side to said trailing side.

20. The method of claim 19, wherein said first and second apertures are coaxially aligned with one another.
